(19) United States
(12) Patent Application Publication
(10) Pub. No.: US 2010/0233168 A1
(43) Pub. Date: Sep. 16, 2010

(54) Rationale for IL-1 Beta targeted therapy in sickle cell disease for ischemia-reperfusion induced complications

(76) Inventor: Alan Wanderer, Bozeman, MT (US)

Correspondence Address: ALAN WANDERER, 1360 NELSON ROAD, BOZEMAN, MT, 59718, US

(21) Appl. No.: 12/660,981

(22) Filed: Mar. 9, 2010

Related U.S. Application Data
(60) Provisional application No. 61/209,804, filed on Mar. 11, 2009.

(51) Int. Cl. A61K 39/395; A61P 9/10; A61P 7/00

(52) U.S. Cl. 424/134.1; 424/145.1

(57) ABSTRACT

Sickle cell patients atypically experience exaggerated inflammatory responses to pathogens that normally cause mild respiratory infections in non-sickle cell humans. There appears to be heightened inflammatory responses to pathogens in combination with hypoxia in sickle cell disease. The novelty of this invention provides a new paradigm to explain the exaggerated inflammatory response of sickle cell disease to pathogens especially when accompanied by hypoxic stress. In particular, sickle cell chest injury and other complications associated with ischemia-reperfusion injury caused by vaso-occlusion can involve co-stimulation of the NALP-3 inflammasome by pathogen associated molecular patterns (PAMPs) and hypoxic-induced danger associated molecular patterns (DAMPs), leading to exaggerated pro-inflammatory responses marked by increased IL-1β secretion and subsequent induction of neutrophilic inflammation. This invention thereby provides the immunologic, biologic and biochemical rationale for IL-1β targeted therapies in sickle cell disease to block the pathological effects of IL-1β that leads to exaggerated inflammatory expressions, including neutrophilic inflammation.

This non-provisional United States patent application claims the benefit of the provisional application U.S. Application No. 61/209,804, filed on Mar. 11, 2009.

### BIBLIOGRAPHY

Gladwin M T, Vichinsky E. Mechanisms of disease: pulmonary complications of sickle cell disease. N Engl J Med 2008; 359: 2254-65.
Belcher J D, Marker P H, Weber J P, Hebbel R P, Vercellotti G M. Activated monocytes in sickle cell disease: potential role in the activation of vascular endothelium and vaso-occlusion. Blood 2000; 96: 2451-9.
Frenette P S. Sickle cell vaso-occlusion: multistep and multicellular paradigm. Curr Opin Hematol 2002; 9: 101-6.
Wanderer A A. Ischemic-reperfusion syndromes: Biochemical and immunologic rationale for IL-1 targeted therapy. Clin Immunol. 2008; 128, 127-32.
Dinarello C A. Mutations in cryopyrin: bypassing roadblocks in the caspasel inflammasome for interleukin-1 β secretion and disease activity, Arthritis Rheum. 2007; 56:2817-2822.
Martinon F, Burns K, Tschopp J. The inflammasome: a molecular platform triggering activation of inflammatory caspases and processing of proIL-1beta. Mol. Cell 2002; 10: 417-26.
Chen C J, Shi Y, Hearn A, Fitzgerald K, Golenbock D, Reed G, Akira S, Rock K L. MyD88-dependent IL-1 receptor signaling is essential for gouty inflammation stimulated by monosodium urate crystals, J. Clin. Invest. 2006; 116:2262-2271.
Shi Y, Evans J E, Rock K L. Molecular identification of a danger signal that alerts the immune system to dying cell. Nature 2003; 425: 516-521.
Hoffman H M, Wright F M, Broide D H, Wanderer A A, Kolodner R D. Identification of a locus on chromosome 1q44 for familial cold urticaria, Am. J. Hum. Genet. 2000; 66:1693-98.
Holtzclaw J D, Jack D, Aguayo S M, Eckman J R, Roman J, Hsu LL. Enhanced pulmonary and systemic response to endotoxin in transgenic sickle mice. Am J Respir Crit Care Med. 2004; 169: 687-95.
Kaul D K, Hebbel R P. Hypoxia/reoxygenation causes inflammatory responses in transgenic sickle mice but not in normal mice. J Clin Invest 2000; 106: 411-20.
Giamarellos-Bourboulis E J, Mouktaroudi M, Bodar E, van der Ven, J, Kullberg B-J, Netea M G, van der Meer J W M. Crystals of monosodium urate monohydrate enhance LPS-induced release of IL-1 by mononuclear cells through a caspase-1 mediated process. Ann Rheum Dis. 2009; 68:273-8.
Church L D, McDermott M F. Canakinumab, a fully-human mAB against IL-1 beta for the potential treatment of inflammatory disorders. Curr Opin Mol Ther. 2009; 11: 81-9.

### BACKGROUND OF THE INVENTION

Sickle cell disease is an autosomal recessive inherited hemoglobinopathy. It is a common autosomal recessive disease affecting 1-4% of infants born in sub-Saharan Africa and affects 1 out of 600 Black-Americans. In the homozygous form, two copies of a mutant β globin gene are inherited causing valine substitution for glutamic acid in the globin chain of hemoglobin A. The amino acid exchange causes the altered hemoglobin (referred to as hemoglobin S) to polymerize on deoxygenation which induces structural damage in red cell membranes, thereby distorting the red cell shape into a sickle appearance. As a consequence rheologic properties of sickle red blood cells impair blood flow through small blood vessels which in turn leads to vaso-occlusion from a combination of entrapped sickle cells, polymerized free sickle hemoglobin, embolized fat cells from bone marrow, activated monocytes, and neutrophils adherent to activated endothelial cells. The ischemia from micro vaso-occlusion can have multiple clinical representations, such as acute chest injury, systemic and pulmonary hypertension, chronic renal failure, strokes, avascular necrosis of bone, leg ulcerations and retinopathy. The recurring vaso-occlusive crises causing ischemic-reperfusion injury (IRI) that occurs in sickle disease is often associated with severe pain, inflammation, fever, and neutrophilic leukocytosis, all of which are hallmarks of sickle cell crises.

The most common cause of lung inflammation associated with sickle cell crises is acute chest injury. Manifestations include pulmonary infiltrates suggestive of alveolar consolidation, fever, respiratory distress, cough and wheezing. It is the most common cause of death in patients hospitalized with sickle cell disease. The causes of acute chest injury are multiple and frequently include exaggerated neutrophilic inflammation from pulmonary infection, intravascular occlusion that can lead to pulmonary infarction, and embolized fat emboli. A consequence of this patho-physiology can be acute pulmonary hypertension, severe pulmonary inflammation and hypoxemia, all of which cause the high mortality associated with this complication. It has been observed that sickle cell patients have exaggerated responses to community acquired pathogens. However, there is no known immune defect associated with sickle disease to explain this clinical phenomenon. Transgenic sickle mice have an increased susceptibility to inflammation when exposed to lipopolysaccharide (LPS), a bacterial endotoxin product, in combination with hypoxia. Transgenic mice also develop lung injury at lower doses of endotoxin and hypoxia that ordinarily do not affect normal wild mice, supporting the observation that in the presence of a hypoxic stimulus, there is unexplained exaggerated inflammation to microbes in sickle cell disease. One object of this patent is to provide an explanation for this clinical phenomenon.

Acute hemolysis with a subsequent fall in hemoglobin concentrations is another patho-physiologic feature that can accompany vaso-occlusion with acute chest injury. Acute anemia can add to severe hypoxemia noted during sickle crises. Hemolysis is associated with extra-cellular polymerized sickle hemoglobin which in turn adds to vaso-occlusion. Additionally rapid hemolysis with catabolism of hemoglobin and nucleotide breakdown from tissues injured by ischemic-hypoxic stress can lead to hyper-uricemia, which in itself can cause more tissue and organ inflammation, as will be detailed.

The current therapy for acute chest injury and other manifestations of acute sickle crises is limited in reversing the patho-physiology of this disorder. Current therapeutic approaches include exchange transfusions, hydroxyurea, and experimental therapies with haptoglobin infusions, inhaled carbon monoxide gas, and carbon monoxide releasing agents. To date there is a need for new therapeutic agents to reduce the complications of acute chest injury and other complications of sickle disease. In other examples of ischemic-reperfusion injury (IRI), innate immune mechanisms play a significant role in causing neutrophilic dominant inflammation. Cytokine Interleukin-1β (IL-1 β) has been particularly implicated in the causation of IRI based on evidence of its increased secretion in animal IRI models. it is known that IL-1 β secretion can occur following pathogen associated molecular pattern (PAMPs) stimulation of innate immune receptors, such as cytoplasmic cryopyrin-inflammasome (NALP-3), the cytoplasmic NALP-1 inflammasome, and cell membrane toll-like receptors (TLRs'). In healthy animal models of IRI, it is unlikely that PAMPs stimulate IL-1β secretion, as release of cytokines occur temporally following blood vessel occlusion. Absent the involvement of PAMPs, it has been posited that biochemical events associated with IRI, such as hypoxia and metabolic acidosis, lead to necrosis of cells with formation of danger associated molecular patterns (DAMPs), i.e. uric acid/urate (UA) and calcium pyrophosphate (CaPP) crystallization, extracellular ATP and intracellular hypokalemia. DAMPs in turn are known to stimulate IL-1β secretion by the NALP-3 inflammasome. Hence in IRI the NALP-3 inflammasome could function as innate immune sentries that are capable of detecting by-products of stressed and dying cells.

Since the NALP-3 inflammasome may be involved in IRI pathogenesis, the following is a short review of salient investigations involving this multi-protein innate cytoplasmic structure. The cryopyrin-encoding gene (C1AS1/NLRP3) on chromosome 1g44 was discovered by investigating Familial Cold Auto-Inflammatory Syndrome (FCAS), a rare auto-inflammatory, autosomal dominant syndrome characterized by cold-induction of IL-1 β secretion that causes fever, neutrophilic leukocytosis, and neutrophilic leukocyte infiltrated dermatosis. The cryopyrin gene discovery led to recognition of the NALP-3 inflammasome which is comprised of a cytoplasmic macromolecular protein complex containing cryopyrin and other adaptor proteins. Two other rare and seemingly unrelated autosomal dominant periodic fever syndromes (i.e. Muckle-Wells and Neonatal Onset Multisystem Inflammatory Disease, i.e. NOMID) with dysregulated 1L-1 β production were found to have mutations on the same gene. All three auto-inflammatory hereditary syndromes referred to as cryopyrin-associated periodic syndromes (CAPS) are exceptionally responsive to IL-1 β targeted therapy (IL-1β TT) as this therapy eliminates neutrophilic inflammation and many of the associated symptoms. This data provided unequivocal evidence for IL-1 β mediation of dominant neutrophilic inflammation in human disorders. Subsequently, investigators recognized that the NALP-3 inflammasome can be stimulated by DAMPs and/or PAMPs to secrete IL-1β. Release of IL-1 β in turn causes a cascade of pro-inflammatory molecular events, such as up-regulation of vascular adhesion molecules (ICAM), IL-6 release, increased neutrophil and monocyte chemokines, IL-17 A secretion, and stimulation of phospholipase-A2 activation with increased formation of LTB₄, all of which can promote and magnify neutrophilic inflammation.

As previously mentioned, sickle cell patients are atypical in that they experience exaggerated inflammatory responses to pathogens that normally cause mild respiratory infections. Observations indicate transgenic sickle mice compared to wild mice require lower concentrations of lipopolysaccharide (LPS) to cause lung injury and increased secretion of IL-1 β and TNFα in bronchoalveolar lavage fluid. A similar exaggerated inflammatory response was noted in hypoxia murine studies, as neutrophilia and activated adherent leukocytes were significantly greater in transgenic sickle mice compared to wild type mice. Hence there appears to be heightened inflammatory responses to PAMPs and hypoxia in sickle cell disease and in transgenic sickle mice.

### BRIEF SUMMARY OF THE INVENTION

Sickle cell patients are atypical in that they experience exaggerated inflammatory responses to pathogens that normally cause mild respiratory infections in non-sickle cell humans. In particular there appears to be heightened inflammatory responses to pathogens and hypoxia in sickle cell disease. The novelty of this invention is to provide a new paradigm to explain the exaggerated inflammatory response of sickle cell disease to pathogens especially when accompanied by hypoxic stress. In particular, sickle cell chest injury and other complications associated with IRI caused by vaso-occlusion may involve co-stimulation of the NALP-3 inflammasome by pathogen associated molecular patterns (PAMPs) and hypoxic-induced danger associated molecular patterns (DAMPs), leading to exaggerated pro-inflammatory responses marked by increased IL-1β secretion and subsequent induction of neutrophilic inflammation.

A second object of this patent is to provide the immunologic, biologic and biochemical rationale for IL-1 β targeted therapies (IL-1βTT) in sickle cell disease to block the pathological effects of IL-1 β that leads to exaggerated inflammatory expressions, and in particular neutrophilic inflammation.

### BRIEF DESCRIPTION OF DRAWING

FIG. 1: composite of sickle cell disease patho-physiology causing exaggerated inflammation

### DETAILED DESCRIPTION OF THE INVENTION

The evidence to explain the novelty of this invention is based on data gathered from an exhaustive review of the literature. The salient data is outlined as follows:
(a) Therapeutic control of CAPS with IL-1β TT provides the scientific support that IL-1β is a profoundly significant cytokine capable by itself of setting off an inflammatory cascade with dominant neutrophilic inflammation;
(b) Neutrophilic inflammation is prominent in sickle cell disease and in transgenic sickle mice subjected to hypoxia and/or PAMPs. Compared to wild mice, transgenic sickle mice exhibit a subclinical pro-inflammatory state marked by elevated neutrophil counts and increased soluble vascular adhesion molecules. In the same studies, following LPS challenge there is heightened inflammatory response in the transgenic sickle mice as compared to wild mice in terms of increased cytokine expression, airway tone and death.
(c) Monocytes from sickle cell anemia patients incubated with endothelial cells caused an increase expression of endothelial adhesion molecules as compared to normal monocytes. Antibodies to IL-1β and TNF-α blocked activation of the endothelium by monocytes. These studies indicate that IL-1 β and TNF-α activate inflammatory endothelial responses and that there are mechanisms intrinsic to sickle cell monocytes that enhance endothelial cell activation. This provides further support for the concept that sickle cell disease has heightened pro-inflammatory responses.
(d) There is convincing evidence for the fundamental role of IL-1 cytokines in IRI as IL-1 α/β knock-out animals exhibit marked reduction of IRI and conversely IL-1 receptor antagonist (IL-1ra) knock-out animals exhibit increased IRI;
(e) Uric acid elevations are frequently observed in sickle cell disease, probably due to a combination of hemolysis and nucleotide breakdown in tissues affected by IRI, leading to hypoxia and metabolic acidosis. Uric acid and urates are particularly insoluble in acidosis as the solubility of urates is 1 to 4 mg/dL in a pH range of 3 to 6 versus 15 mg/dl or higher in pH>7.0. The presence of high uric acid in combination with focal ischemic induced metabolic acidosis provides optimal milieu for crystallization of urates which can stimulate the NALP-3 inflammasome;
(f) Studies support synergistic IL-1β secretion from human monocytes co-stimulated by LPS and urate crystals. The synergy between urate crystals and LPS was directed at the level of the NALP3 inflammasome.

When observations (a) thru (f) are considered individually they do not by themselves explain the exaggerated inflammatory response associated with infections and hypoxia in sickle cell crises. However this phenomenon can be explained when considering all data together.
Observation (a) indicates that robust neutrophilic inflammation can occur from IL-1 beta secretion;
Observations (b) and (c) indicate that neutrophilic inflammation and neutrophilia are prominent in sickle cell crises. Moreover monocytes from sickle cell anemia patients incubated with endothelial cells cause an increase expression of endothelial adhesion molecules as compared to normal monocytes and antibodies to IL-1β and TNF-α blocked activation of the endothelium Increased expression of endothelial adhesion molecules by IL-1β in turn cause neutrophils to adhere to the intima of the micro-vasculature, and thereby enhance vaso-occlusion and IRI. In addition as previously described, the observations in CAPs indicate that IL-1 β can induce a cascade of pro-inflammatory molecular events leading to prominent neutrophilic inflammation.
Observation (d) provides further support for the role of IL-1 β in the causation of inflammation associated with IRI, a pathophysiology that occurs in sickle cell disease occurring from the propensity of vaso-occlusion in this disorder;
Observation (e), indicates that DAMPs such as urate crystals, and PAMPs can stimulate secretion of IL-1β from the NALP-3 inflammasome. Furthermore, hyperuricemia occurs in patients with sickle cell crises from a combination of hemolysis and necrosis of tissues resulting from vaso-occlusion, ischemia- and hypoxic induced tissue injury;
Observation (f) indicates that synergistic secretion of IL-1β secretion occurs in human monocytes co-stimulated by LPS and urate crystals. This synergy at the level of the NALP3 inflammasome can explain the exaggerated inflammatory responses seen in hypoxic stressed sickle cell patients affected by microbial infections.

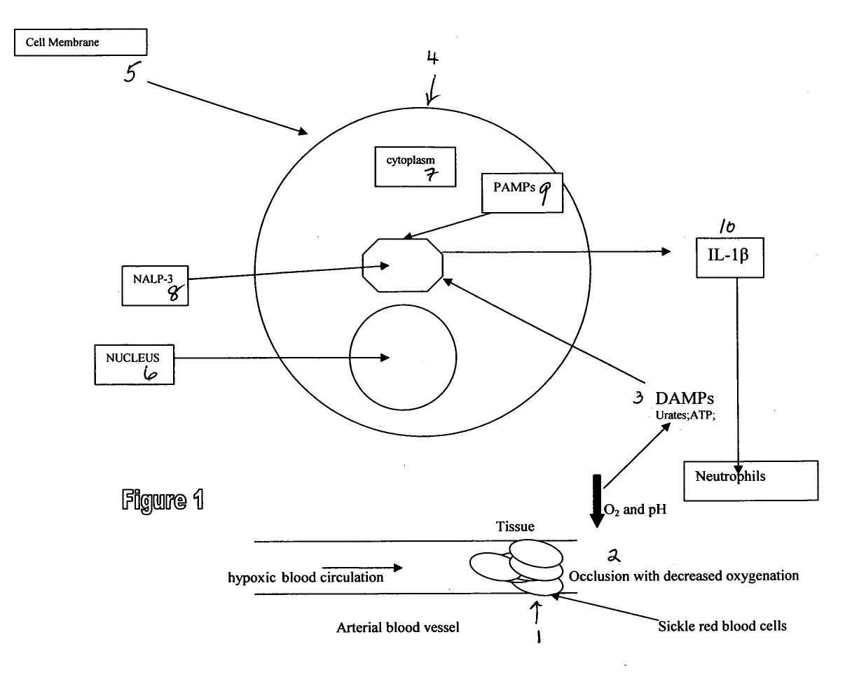

All together, these observations support the novelty of this invention, which is described in detail by FIG. 1 as follows:

Sickle cell disease patients exhibit vaso-occlusion from aggregated sickle cells(1) caused by systemic hypoxemia associated with lung infection or acute chest injury. The hypoxemia causes sickle red blood cells to elongate into a sickle shape leading to aggregation and clotting in small blood vessels. Added to the aggregated sickle cells are platelets, neutrophils, free sickle hemoglobin and free fat cells, none of which are shown. Distal to the clot (2) there is ischemia, increased hypoxia and metabolic acidosis in adjacent tissue receiving reduced oxygen diffusing through capillaries. There is concomitant breakdown of cells, nucleotides, freeing of ATP, intracellular hypokalemia, dissolution of cells and buildup of urates and calcium pyrophosphates which crystallize at low pH. All of the aforementioned biochemical responses are referred to as DAMPs (3). An enlarged cell (4) is shown with a cell membrane (5), nucleus (6), and cytoplasm (7) which contains the aggregated NALP-3 inflammasome (8). PAMPs (9) are shown inside the cytoplasm (7). The DAMPs (3) such as crystallized urates or free ATP are detected by the cell membrane (5) and/or by other as yet undetermined means) and their presence in turn stimulate the NALP-3 inflammasome (8). IL-1β (10) is stored in cytoplasmic (7) organelles as pro-IL-1β and stimulation of the NALP-3 inflammasome by DAMPs (3) and/or PAMPs (9), cause the pro-IL-1β to be catalyzed to IL-1β (10), allowing it to be secreted into extracellular fluids. The secreted IL-1β (10) in turn cause attraction of neutrophils via a cascade of previously described immunologic events, such as by attracting chemokines and by increased vascular adhesion molecule activation which in turn cause neutrophils to adhere to intima of blood vessels. The buildup of neutrophils cause many inflammatory events such as tissue damage by release of neutrophil proteases which damage cell and tissue integrity. PAMPs (9) in combination with DAMPs (3) or PAMPs(9) or DAMPs (3) by themselves can stimulate innate immune receptors such as the NALP-3 Inflammasome (8) and even certain toll-like receptors (TLR 2 and TLR-4) to secrete IL-1β (10). Moreover the co-stimulation of the NALP3-inflammasome (8) by DAMPs (3) and PAMPs (9) can cause synergistic IL-1 β secretion, leading to greater inflammation. Altogether there is a heightening of inflammation associated with sickle cell crises with high unremitting fever, increased hypoxia, and more neutrophilia. Eventually respiratory and cardiovascular collapse occur leading to shock and death. The aforementioned concept provides an explanation for sickle cell exaggerated inflammatory responses to the combination of microbial infection and/or hypoxic effects from IRI.

Currently there has been no known or published use of IL-1β targeted therapy (IL-1β TT) to minimize complications in sickle cell crises caused by IL-1β induced neutrophilic inflammation. In part this has occurred because many other inflammatory molecules have been targeted in inflammatory conditions, such as anti-TNF α, which can cause serious super-infections and an increase incidence of malignancies. The concept proposed in this patent now provides the impetus and the biologic, biochemical and immunologic rationale to use this therapy in this condition. It is suggested that sickle cell acute chest injury as well as other IRI complications could benefit with blockade of IL-1 β biologic effects using any of several commercially available biologics, such as an IL-1 β receptor blocker (anakinra; Kineret®), IL-1 β TRAP (rilanocept; Arcalyst®), and monoclonal anti-IL-1 β antibodies (canakinumab by Novartis). These biologics have been used for many years in treatment of CAPS and have been efficacious with excellent safety profiles. Moreover rilanocept and canakinumab have half lives of a minimum of two weeks. Hence a single injection of these biologics would theoretically be adequate in reducing the pro-inflammatory effects of IL-1 β in acute chest injury and could be used as an adjunct to reduce severity of this and other acute and/or chronic sickle cell associated complications caused by vaso-occlusion and IRI.

In summary the novelty of this invention is the recognition that sickle cell disease with exaggerated inflammatory responses occurs when DAMPs formation from IRI and PAMPs enhance the secretion of IL-1 β by the NALP-3 inflammasome. This provides the rationale to block IL-1β with IL-1 β TT that can include IL-1 β receptor blockers, IL-1 β TRAP, and monoclonal anti-IL-1 β antibodies. In addition it can include any therapeutic intervention that can prevent IL-1 β formation or secretion, such as agents that decrease DAMPs formation, such as uric acid production with allopurinol and its analogs.

I claim:

1. Use of IL-1 β targeted therapy to control exaggerated inflammatory responses associated with sickle cell ischemia-reperfusion injury;

2. The claim that said exaggerated inflammation in claim 1 is caused by formation of danger associated molecular patterns as a consequence of sickle ischemic-reperfusion injury which can stimulate secretion of interleukin-1 β from innate immune receptors;

3. The claim in which said exaggerated inflammation in claim 2 is exaggerated because of synergistic stimulation of the NALP-3 inflammasome by pathogens and said danger associated molecular patterns, thereby causing enhanced secretion of interleukin-1 β in sickle cell disease;

4. The claim that IL-1 β targeted therapy in claim 1 can interfere in the biologic action of secreted IL-1 β;

5. The claim in claim 4 that said IL-1 β targeted therapy includes IL-1 β receptor blockers, IL-1 β TRAP, and monoclonal anti-IL-1 β antibodies;

6. The claim in claim 5 that said IL-1 β targeted therapy include therapeutic agents that reduce DAMPs formation that stimulate secretion of IL-1 β from innate immune receptors;

7. The claim that said complications of ischemia reperfusion injury associated with sickle cell disease in claim 1 include acute chest injury;

8. The claim that said complications of ischemia reperfusion injury associated with sickle cell disease in claim 1 include mum-organ complications such as but not limited to stroke, aseptic necrosis of the bones, fat necrosis in bone marrow, and renal dysfunction.

9. The claim in which sickle cell patients exhibit exaggerated inflammation from the combination of pathogen and hypoxic stimulation;

10. The claim in which exaggerated synergistic inflammation in claim 9 is caused by stimulation of innate immune receptors by pathogen associated molecular patterns and danger associated molecular patterns;

11. The claim that said danger associated molecular patterns in claim 10 occur from hypoxic induced tissue injury secondary to sickle cell vaso-occlusion;

12. The claim that said exaggerated inflammation in claim 9 is caused by enhanced secretion of interleukin-1 β causing neutrophilic inflammation;
